(19) United States
(12) Patent Application Publication
(10) Pub. No.: US 2018/0218496 A1
(43) Pub. Date: Aug. 2, 2018

(54) Automatic Detection of Cutaneous Lesions

(71) Applicant: Emerald Medical Applications Ltd., Modiin (IL)

(72) Inventors: Ilan Sinai, Pardes Hanna (IL); Marina Asherov, Rishon Lezion (IL); Lior Wayn, Modiin (IL); Adi Zamir, Kfar Saba (IL)

(73) Assignee: Emerald Medical Applications Ltd., Modiin (IL)

(21) Appl. No.: 15/748,808

(22) PCT Filed: Jul. 28, 2016
(86) PCT No.: PCT/IL2016/050830

§ 371 Date: Jan. 30, 2018

(30) Foreign Application Priority Data
Jul. 30, 2015 (GB) 1513454.7

(51) Int. Cl. G06T 7/00; G06T 5/00; G06T 7/90; G06T 5/20; A61B 5/00

(52) U.S. Cl. CPC G06T 7/0012; G06T 5/008; G06T 7/90; G06T 5/002; G06T 5/20; G06T 2207/10024; A61B 5/0077; A61B 5/448; G06T 2207/30096; G06T 2207/30088; A61B 5/444

(57) ABSTRACT

A computerized system and method for analyzing a digital photograph containing identified skin parts and analyzing and identifying cutaneous lesions. The method comprises: enhancing lesions in the identified skin parts; detecting hair patches; approximating localization of all lesions, and identifying lesions pixels.

### TECHNICAL FIELD

The present invention relates to image analysis in general, and in particular to analyzing a photograph of a person and detecting cutaneous lesions.

### BACKGROUND ART

Skin cancer is unfortunately a source of great concern, in particular but note exclusively for people long exposures to the sun in hot places. As with most diseases, early detection is key in increasing the chances to overcome the cancer.

Nowadays, digital cameras and mobile phones equipped with digital cameras are increasingly popular. It is thus very easy for most people to take pictures of themselves with exposed skin parts. The problem is that only a dermatologist would know to look at the lesion and diagnose whether it's benign or not.

There is thus a need for an application, accessible via a mobile phone or a personal computer, that would analyze a digital photograph of a person and not only identify lesions but recommend possible next steps if relevant.

### SUMMARY OF INVENTION

It is an object of the present invention to provide a system and method for identification of cutaneous lesions on a digital photograph.

It is another object of the present invention to provide a system and method for counting cutaneous lesions on a digital photograph and identifying their position.

It is a further object of the present invention to provide a system and method for counting cutaneous lesions on a digital photograph of people with different skin colors.

It is yet another object of the present invention to provide a system and method for counting cutaneous lesions on a digital photograph in both hairy and non-hairy human skin parts.

The present invention relates to a computing system comprising at least one processor; and at least one memory communicatively coupled to the at least one processor comprising computer-readable instructions that when executed by the at least one processor cause the computing system to implement a method for analyzing a digital photograph comprising identified skin parts and analyzing cutaneous lesions, the method comprising the steps of:
(i) enhancing lesions in said identified skin parts, wherein said enhancing lesions comprises the steps of:
a. detecting skin complexion using common/averaged value of density estimation on a dominant channel extracted from skin pixels;
b. boosting lesions pixels by enhancement of lesion pixels and suppression of skin pixels; and
c. enhancing said Dominant Channel by combining said Dominant Channel with lesions boosting mechanism;
(ii) detecting hair patches;
(iii) approximating localization of all lesions; and
(iv) identifying lesions pixels.

In some embodiments, the dominant channel is saturation, value, intensity, Red Green Blue (RGB) or any combination thereof.

In some embodiments, detecting hair patches comprises the steps of:
(i) calculating one or more hair detection filters based on enhanced dominant channel (EDC);
(ii) calculating local normalized median or average on the filtered EDC;
(iii) calculating density estimation on the “value-EDC” planes or other planes;
(iv) detecting clusters;
(v) calculating how close is each cluster to hair color and skin color and assigning a hair color score each cluster; and
(vi) assigning a patch hair probability score to each cluster based on each cluster's hair color score and savannah score.

In some embodiments, detecting clusters is performed using semi-supervised k-means or spectral clustering or any other clustering method.

In some embodiments, approximating localization of all lesions comprises the steps of:
(i) calculating one or more edge detection filters on EDC plane, said filters varying in length and coefficients values;
(ii) calculating of local median; average; median and standard deviation; or average and standard deviation on the filtered magnitude EDC image;
(iii) combining the result of step (i) and (ii) to create an automatic threshold setting for segmentation for each and every pixel on all regions and for every filter;
(iv) combining said various pixels outcomes and filters decisions to a objects candidates map;

(v) cleaning, smoothing and unifying objects based on filters and proximity;
(vi) filling small holes and gaps;
(vii) removing candidates that are not fully shown in a skin region or in entire image;
(viii) removing candidates that are too small, too narrow or too lacy; and
(ix) cleaning, smoothing and unifying objects again based on morphological filters.

In some embodiments, the edge detection filters are of different shapes, sizes and structures based partly on patch hair probability scores.

In some embodiments, the morphological filters are operations to clean, smooth and remove small blobs and consolidate blobs.

In some embodiments, the morphological filters size is A*B, where A and B are a number between 1-15.

In some embodiments, identifying of lesion pixels comprises performing the following steps for each lesion candidate:

(i) taking from image planes red/green/blue/value/EDC or any combination of one or more of said image planes the pixels that include the lesion candidate as well as its neighboring pixels;
(ii) performing density estimation and maximization of the inter class variation in order to get a suggested threshold for accurate segmentation;
(iii) verifying that the suggested threshold from (ii) is within a defined range;
(iv) perform thresholding, thus creating candidate objects;
(v) cleaning, smoothing and unifying objects based on morphological filters and proximity; and
(vi) fill small holes and gaps.

In some embodiments, identifying of lesion pixels further comprising the step of removing candidates based on one or more morphological features.

identifying of lesion pixels one or more morphological features comprise: Area, Elongation, Euler number, Eccentricity, Major Axis Length, Convex ratio, Convex area, normalized Extent, Extent, normalized Solidity, Solidity.

In some embodiments, the digital photograph was taken according to a total body photography protocol.

In some embodiments, the lesions detected are of 0.5 millimeter (mm) or bigger.

In another aspect, the present invention relates to a computer system composing a processor; and a memory communicatively coupled to the processor comprising computer-readable instructions that when executed by the processor cause the computer system to execute instructions for analyzing a digital photograph comprising identified skin parts and analyzing cutaneous lesions, the system comprising:
(i) an enhancement module adapted to enhancing via the processor lesions in said identified skin parts, wherein said enhancing lesions comprises the steps of:
a. detecting skin complexion using common/averaged value of density estimation on a dominant channel extracted from skin pixels;
b. boosting lesions pixels by enhancement of lesion pixels and suppression of skin pixels; and
c. enhancing said Dominant Channel by combining said Dominant Channel with lesions boosting mechanism;
(ii) a detection module adapted for detecting via the processor hair patches;
(iii) an approximation module adapted for approximating via the processor localization of all lesions; and
(iv) an identification module adapted for identifying via the processor lesions pixels.

### BRIEF DESCRIPTION OF DRAWINGS

FIGS. 1A-1B show an example of a digital photograph (FIG. 1A) and then after enhanced saturation (FIG. 1B).

FIGS. 2A-2C Enhanced Dominant Channel (EDC) Creation. FIG. 2A shows a Dominant channel extracted from digital photograph with a visible lesion at the center. FIG. 2B shows a distribution of skin pixels. Most of the pixels are non-lesions while the minority are lesions. FIG. 2C shows the same image of FIG. 2A with boosted lesion pixels surrounded by suppressed skin pixels. In FIG. 2B, the X axis shows increasing intensity values, and the Y axis shows the number of pixels.

FIGS. 3A-3B illustrate an example of hair detection. FIG. 3A is a digital photograph of a torso with hair patches. FIG. 3B shows the same photograph of FIG. 3A after segmented hair detection filters are applied on ECD, Hairy patches are shown in white. Axes are image coordinates.

FIGS. 4A-4B illustrate the “Savannah Score” feature. Sum of Filter responses on each rectangle on FIG. 4B is shown in FIG. 4A coded in colors. Compare a “hairy” rectangle (Green) to “non-hairy” rectangle (Orange). FIG. 4B axes are image coordinates. FIG. 4A axes are rectangles numbering along the X & Y axes. The rectangles can be shown in FIG. 4A.

FIGS. 5A-B illustrate the “Hair color Proximity” feature on two lesions. FIG. 5A illustrates density distribution of Hair “lesion”. FIG. 5B illustrates density distribution of skin lesion. Red parallelogram shows Hair color anchor. The gray circle shows bare skin anchor. Axes are increasing Saturation and Value values from right to left and down to top. Colors resemble density estimation.

FIG. 6A is a digital photograph of the back of a person, showing two tattoos. FIGS. 6B-6C illustrate the outcomes of two edge filters and their segmentations results.

FIG. 7 shows the results of fusion of multiple segmented filters. Axes are image coordinates.

FIG. 8A is a digital photograph of the back of a person, showing two tattoos. FIG. 8B shows the results of cleaning, smoothing and unifying objects.

FIGS. 9A-9D show the results of holes filling. FIG. 9A shows mask input, FIG. 9B shows masked filled with small holes, non-filled holes can be seen in FIG. 9C, and filled holes are shown in FIG. 9D.

FIG. 10 shows the results of removal of candidates that are not fully shown in the frame region of interest.

FIG. 11A shows a man's naked back with two tattoos, while FIG. 11B shows the results of the 2nd cleaning, smoothing and unifying objects.

FIG. 12A shows a close up of the naked back showing one tattoo, while FIG. 12B shows a sample outcome of the approximate localization process.

FIGS. 13A-13B show two candidates: Candidate 106 is an actual mole while candidate 114 is a FP muscle wrinkle.

FIGS. 14A-C shows the identification process of Candidate 106 (actual mole) and candidate 114 (FP muscle wrinkle) of FIGS. 13A-13B. FIG. 14A is the RGB input. FIG. 14B is the approximate segmentation performed in previous steps. FIG. 14C is the accurate segmentation done in this step.

FIGS. 15A-C shows the identification process of Candidate 106 (actual mole) and candidate 114 (FP muscle wrinkle) of FIGS. 13A-13B. FIG. 15A is the RGB input. FIG. 15B is the approximate segmentation performed in previous steps. FIG. 15C is the accurate segmentation done in this step.

FIG. 16A shows an outcome of all detected lesions. All detections are superimposed as green contours on the original image.

FIG. 16B shows a zoom-in of certain detected lesions of FIG. 16A.

FIG. 17 is a shape for explain morphological operations.

FIG. 18 is an example of pictures of a person taken according to the Total Body Pictography (TBP) protocol.

### MODES FOR CARRYING OUT THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

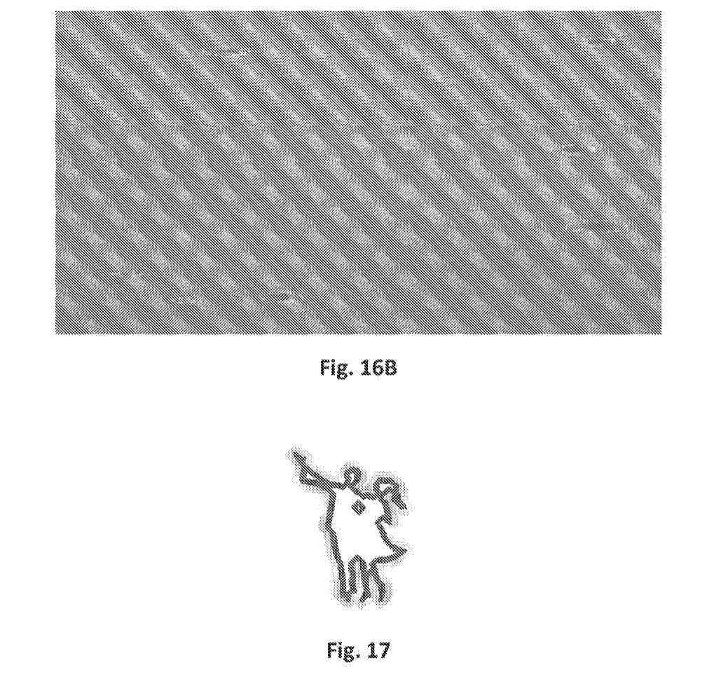

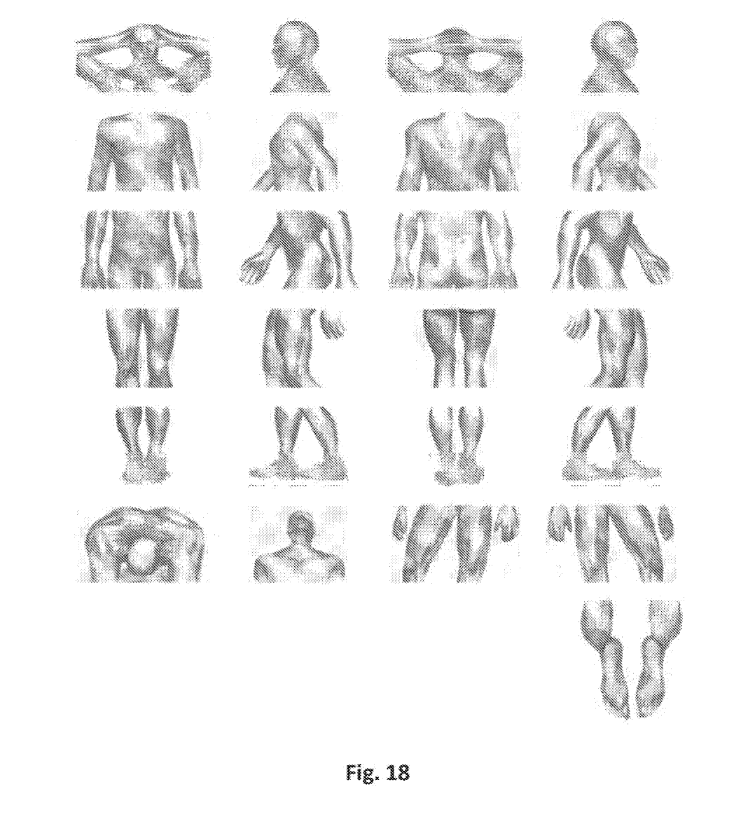

### GLOSSARY

| Term | Definition |
|---|---|
| Object/Class/ | Set of pixels belonging to same entity. Theses pixels |
| tag/category | were grouped together as the result of the |
| | segmentation and classification processes. |
| Segmentation | In computer vision, image segmentation is the process |
| | of partitioning a digital image into multiple segments |
| | (sets of pixels). The goal of segmentation is to |
| | simplify and/or change the representation of an image |
| | into something that is more meaningful and easier to |
| | analyze. Image segmentation is typically used to locate |
| | objects and boundaries (lines, curves, etc.) in images. |
| | More precisely, image segmentation is the process of |
| | assigning a label to every pixel in an image such that |
| | pixets with the same label share certain characteristics. |
| Classification | In machine learning and statistics, classification is the |
| | problem of identifying to which of a set |
| | of categories (sub-populations) an object belongs. An |
| | example would be assigning a given email into “spam” |
| | or “non-spam” classes or assigning a diagnosis to a |
| | given patient as described by observed characteristics |
| | of the patient (gender, blood pressure, presence or |
| | absence of certain symptoms, etc.). |
| | In this context, the objective is to identify and decide |
| | which label (found in the segmentation process) |
| | should be tagged as Skin, background (non-skin) or |
| | others. |
| feature | A feature is an individual measurable property of a |
| | phenomenon being observed/calculated |
| Hard decision | In the Classification process: For each class: Some of |
| | the features contribute to the final classification |
| | decision and some not. |
| Soft Decision | In the Classification process, for each class: All the |
| | features contribute to the final classification decision |
| Dominant channel | Saturation, value, Red-Green-Blue (RGB), any other |
| | channel or any combination thereof. |
| ECD | Enhanced Dominant Channel - Dominant channel |
| | combined with a boosting mechanism. |
| Second channel | Saturation, value, Red-Green-Blue (RGB), any other |
| | channel or any combination thereof. |
| Savannah | Estimated amount of hair in the local skin vicinity |
| Score feature | |
| “Hair color | Estimate how close is the candidate to hair color. |
| Proximity” | |
| Skin Complexity | Most prominent (common) intensity value. Used for |
| | ECD. |
| True Positive | Taking as an example, a study evaluating a new test |
| (TP) | that screens people for a disease. Each person taking |
| False Positive | the test either has or does not have the disease. The |
| (FP) | test outcome can be positive (predicting that the |
| False negative | person has the disease) or negative (predicting that the |
| (FN) | person does not have the disease). The test results for |
| | each subject may or may not match the subject's actual |
| | status. In that setting: |
| | True positive: Sick people correctly diagnosed |
| | as sick |
| | False positive: Healthy people incorrectly |
| | identified as sick |
| | False negative: Sick people incorrectly |
| | identified as healthy |
| | In general, Positive = identified and negative = |
| | rejected. Therefore: |
| | True positive = correctly identified |
| | False positive = incorrectly identified |
| | False negative = incorrectly rejected |
| Morphological | FIG. 17 shows a shape (in blue) and its morphological |
| operations: | dilation (in green) and erosion (in yellow) by a |
| dilation, erosion. | diamond-shape structuring element |
| Morphological | These operation are based on dilation, erosion and |
| operations: | perform: |
| cleaning, | Cleaning - removing small isolated points (“debris”) |
| smoothing, | Smoothing - smooth sharp blob tips. |
| unifying | Unifying - merge several points that are nearby |

The present invention relates to a computerized method comprising a processor and memory for analyzing a digital photograph comprising identified skin parts and analyzing cutaneous lesions apparent in identified skin parts the digital photograph. The method and system of the invention start with a digital photograph where exposed skin parts are already identified.

A digital photograph can initially be in many formats. The two most common image format families today, for Internet images, are raster and vector. Common raster formats include JPEG, GIF, TIFF, BMP, PNG, HDR raster formats etc. Common vector image formats include CGM, Gerber format, SVG etc. Other formats include compound formats (EPS, PDF, PostScript etc.) and Stereo formats (MPO, PNS, JPS etc.).

The first step is to enhance lesions in the identified skin parts. Lesion enhancement comprises several steps:

Skin complexion detection—a common value is extracted from density estimation on a dominant channel. The dominant channel may be saturation, value, Red-Green-Blue (RGB), any other channel or any combination thereof. The skin complexion detection is performed on for all pixels categorized as skin pixels.

EDC (Enhanced dominant channel) creation—combine dominant channel values with boosting/suppression factor for each pixel, resulting in enhancement of lesion pixels and suppression of skin pixels. The boosting is a mathematical function that decrease values of skin pixels and increase values of lesion pixels. See Eq. 1. EDC Channel pixel value (new) is sum of multiplication of the dominant channel pixel value (with or without its neighbors) with Boosting Function F. The Boosting Function F takes into account the Skin complexion value B as well as the “dominant channel” ƒ₁ w/o “Second channel” ƒ₂. See definitions in the glossary. The function can be linear or non-linear (with/without its neighbors). ƒ₂ can be saturation, value, Red-Green-Blue (RGB), any other channel or any combination thereof.

$$EDC formula \\ {f}_{j} ( new ) = {\sum }_{i , j = 1}^{N} {f}_{j} ( old ) * F ( {f}_{i 1} , {f}_{2 j} , B ) \qquad Eq . 1$$

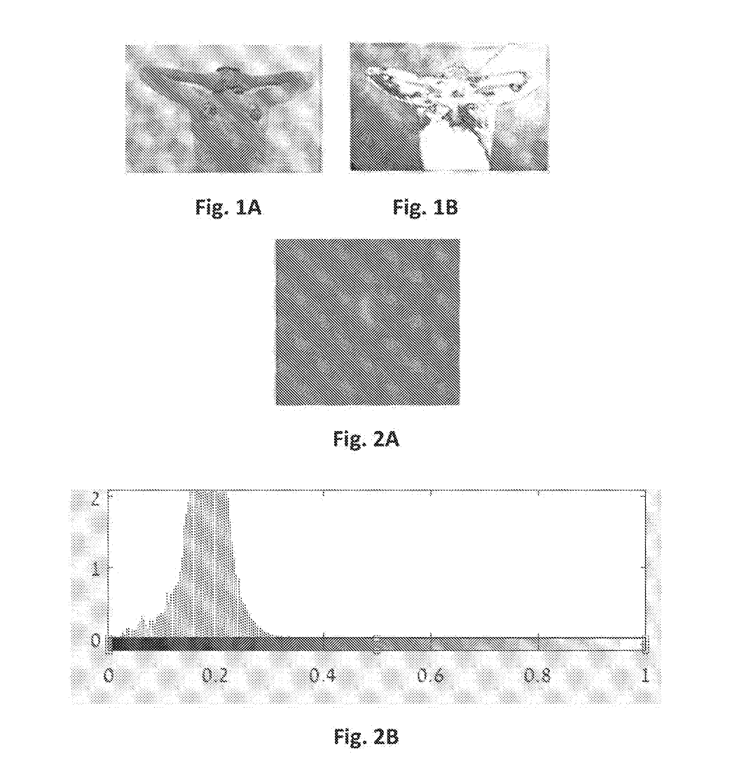

FIGS. 1A-1B show an example of a digital photograph (FIG. 1A) and then the same photograph after applying Enhanced Dominant Channel (ECD) (FIG. 1B). Skin portions are clearly shown in white in FIG. 1B.

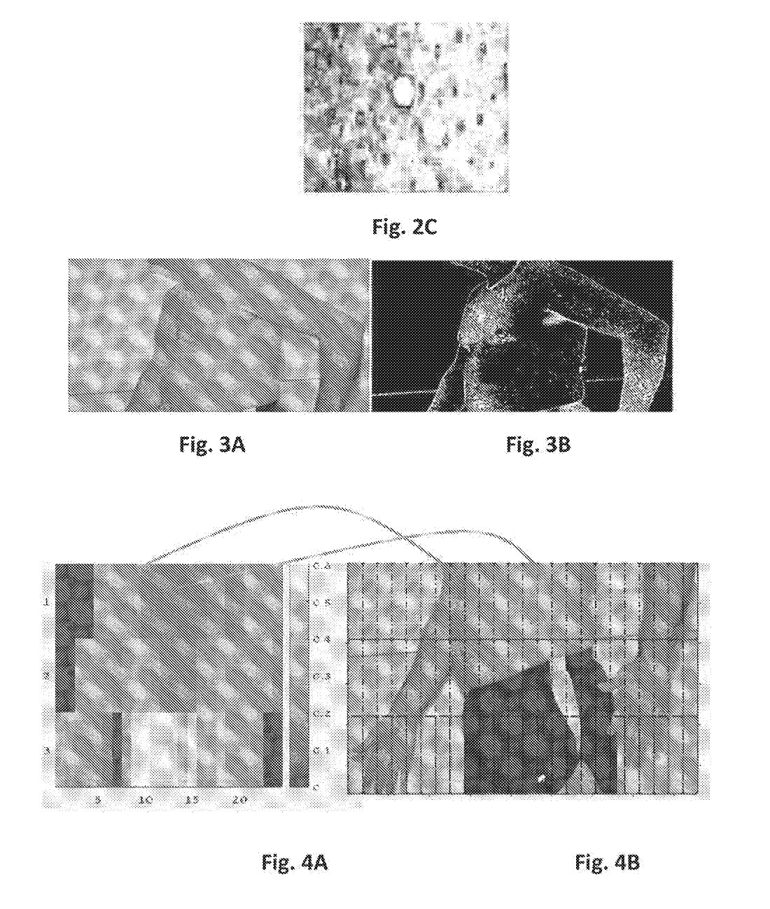

FIGS. 2A-2C shows examples of boosting and suppression processes. FIG. 2B shows a distribution of skin pixels. Most of the pixels are non-lesions while the minority are lesions. FIG. 2A shows a digital photograph dominant channel with a visible lesion at the center, shown as a light square-like figure. FIG. 1C shows the same image of FIG. 2A with boosted lesion pixels surrounded by suppressed skin pixels. This is the ECD.

The next step is detecting hair patches. it is important to detect hair patches as to separate them from bare skin. Detecting hair patches involves the following steps:

Calculation of one or more hair detection filters on ECD plane or dominant channel plane or both.

Savannah feature: calculation of local normalized (relevant pixels only) median and/or average on the filtered ECD or dominant channel filtered image.

Calculation of 3-Dimensional (3D) density estimation on the ECD/Dominant channel and “Second channel” channels.

Cluster detection using semi-supervised k-means, spectral clustering or any other clustering method.

Hair color proximity feature: calculating how close is the main color of each cluster to hair color (black/brown etc.) and skin color.

Hair patches score: fusion of Savannah feature score and hair color proximity score in order to make a decision for each skin region/patch regarding the amount of hair it contains. The decision can also calculate the confidence (probability) of the calculation.

FIGS. 3A-3B illustrate an example of hair patches detection. FIG. 3A is a digital photograph of a torso with hair patches. FIG. 3B shows the same photograph of FIG. 3A after segmented hair patches detection filters are applied on ECD. Hairy patches are shown in white.

FIGS. 4A-4B shows example of “Savannah Score” feature. Sum of Filter responses on each rectangle on FIG. 4B is shown in FIG. 4A coded in colors. Compare a “hairy” rectangle (Green) which has high score to “non-hairy” rectangle (Orange) with relatively hair free with low score of 0.1.

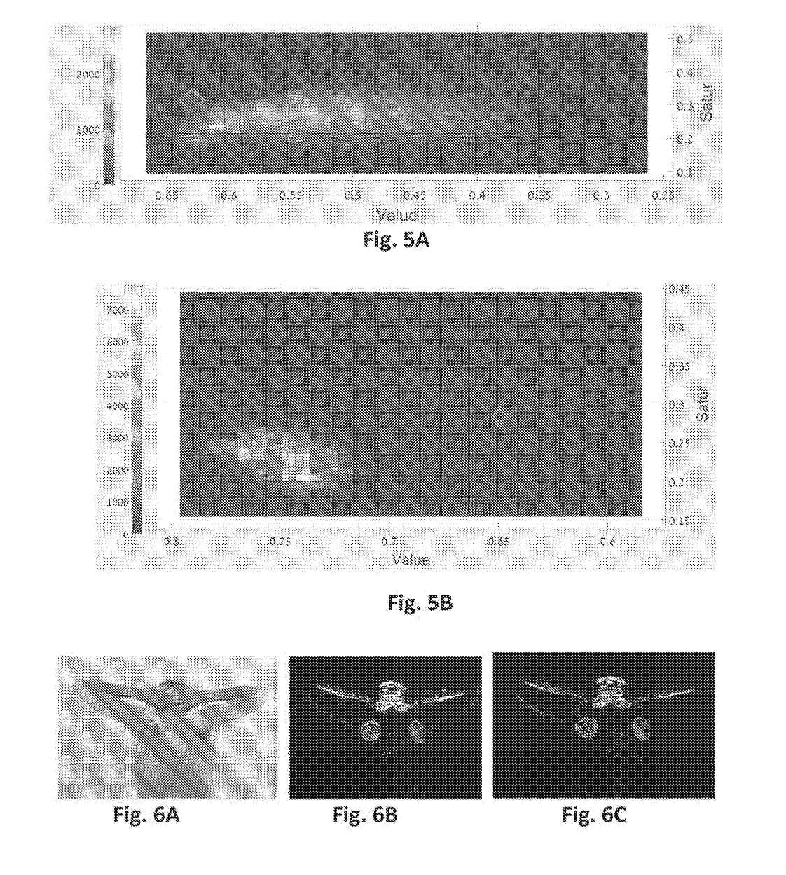

FIGS. 5A-5B shows an example of “Hair color Proximity” feature on two lesions.

FIG. 5A shows density distribution of Hair “lesion”. FIGS. 5B shows density distribution of skin lesion. Red triangle—Hair color anchor. Gray circle—bare skin anchor. Lesion 5A is closer to Hair color anchor (red triangle mark) while lesion 5B is closer to bare skin anchor (gray circle mark).

The next step is an Approximate Localization of all lesions. The approximation comprises the following steps:

Calculating one or more edge detection filters on EDC and Dominant channel planes. The edge detection filters are of different shapes, sizes and structures based partly on patch hair score. Specific regions (parts of the image) can be re-scanned with more sensitive filter, if needed.

Calculating local median, average, standard deviation and any combination thereof on the filtered image.

Combining the results of the previous calculations together with automatic threshold setting for segmentation for various regions and filters.

Fusion of one or more regions and filters decisions to a candidates map. Cleaning, smoothing and unifying objects based on averaging filters said proximity. Filters size can be any number between 1-15 and can one or two dimension, the filters coefficients can be any number between 0 to 1.

For example 6×4 filter can be [1, 1, 1, 1, 1, 1]

1, 1, 0.5, 0.5, 1, 1

1, 1, 0.5, 0.5, 1, 1

1, 1, 1, 1, 1, 1].

Or 9×1 filter of the form [1, 1, 0.75, 0.75, 0, 0.75, 0.75, 1, 1].

Filling small holes and gaps.

Removal of candidates that are not fully shown in our frame region of interest.

Removing candidates that are too small, too narrow, too lacy; this is done by applying set of criteria. For every candidate we calculate set of features and compare them to predefined limiting values. The list of features limiters is given in end of Table 1. The calculated features include, but not only:

Candidate Area, Perimeter, aspect ratio, convex ratio. Number of holes and their respective area. For example, “Minimal/Maximal blob area” is used to filter based on candidate area. Too narrow candidate will be removed with the Maximal Eccentricity and Maximal MajorAxisLengthT limiters.

2nd cleaning, with the smoothing and unifying objects based on averaging filters and proximity.

FIG. 6A is a digital photograph of the back of a person, showing two tattoos. FIGS. 6B-6C illustrate the outcomes of two edge filters and their segmentations results. The filters can have varying size and different kernel values as described in Table 1. For example, first filter can be [2 2 1 −1 −2 −2] while the second filter is longer: [2 2 2 1 −1 −2 −2−2].

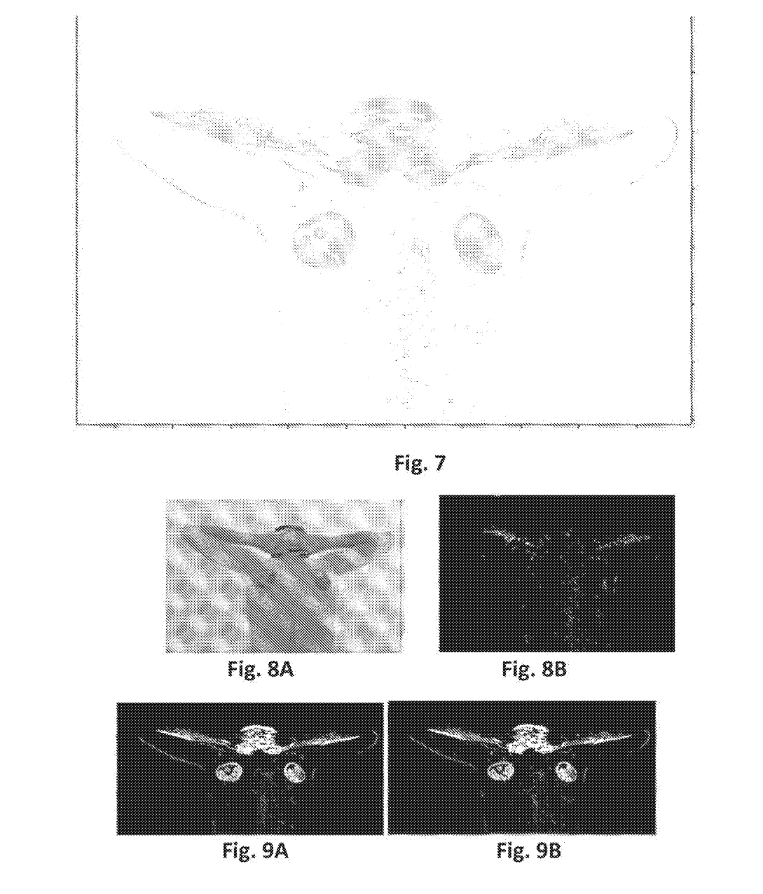

The results of fusion of multiple segmented filters are shown in FIG. 7.

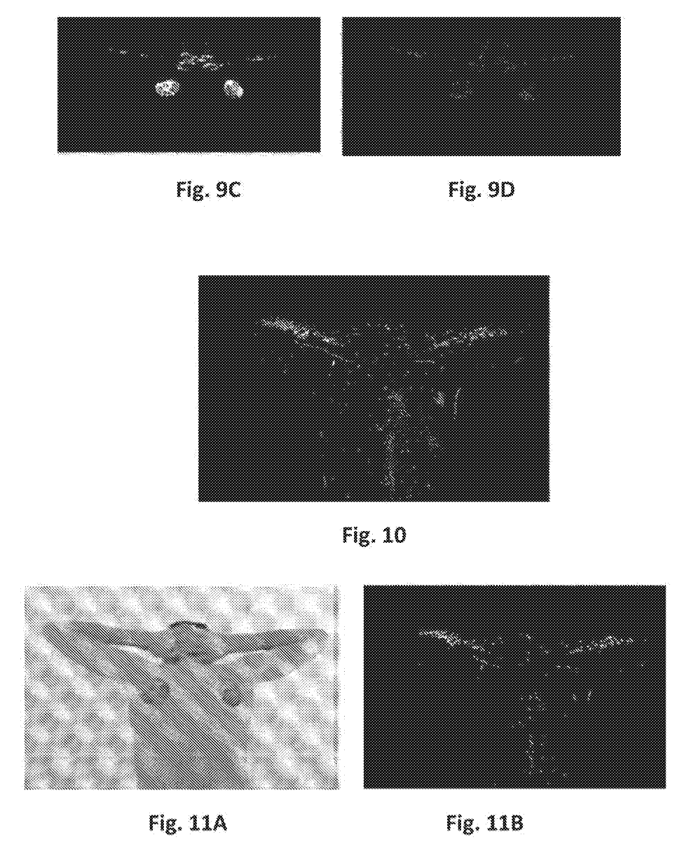

Cleaning, smoothing and unifying objects can be seen in FIG. 8B, while the results of holes filling are shown in FIGS. 9A-9D. FIG. 9A shows mask input, FIG. 9B shows masked filled with small holes, non-filled holes can be seen in FIG. 9C, and holes filed are shown in FIG. 9D.

Removal of candidates that are not fully shown in our frame region of interest is shown in FIG. 10. FIG. 11A shows a man's naked back with two tattoos, while FIG. 11B shows the results of the 2nd cleaning, smoothing and unifying objects. As can be seen, candidates there were too small or too elongated were removed.

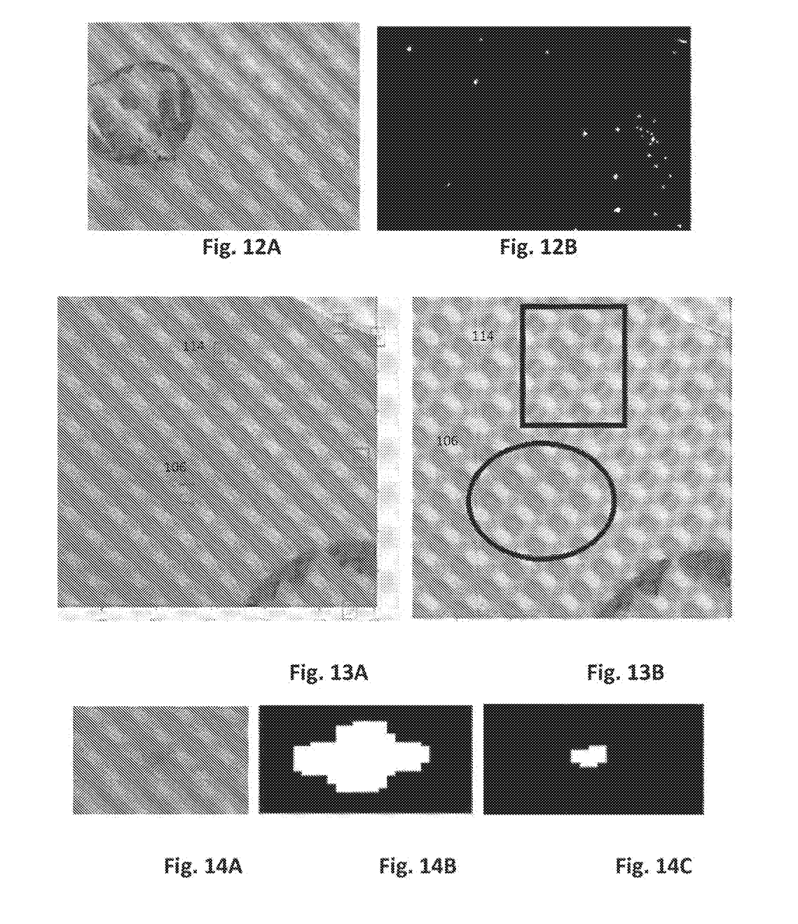

FIG. 12A shows a close up of the naked back showing one tattoo, while FIG. 12B shows a sample outcome of the approximate localization process. This sample shows TP as well as some FP candidates.

The final step involves accurate identifying of lesions pixels.

For each lesion candidate the following steps are performed:

Given the input image planes: Red/green/Blue or any combination of them we extract from the image plane(s) the pixels that include the lesion candidate as well as its immediate surroundings. The immediate surroundings are pixels that are not part of the lesions but reside few (1 to 15) pixels only away from the candidate lesion.

Performing density estimation and maximization of the inter class variation in order to get an accurate segmentation.

Verifying that the resulted threshold is not too low or not too high. We confined the threshold with limiters that are given as parameters beforehand. See table 1.

Cleaning, smoothing and unifying objects based on averaging filters and proximity.

Filling small holes/gaps.

Filtering out, if necessary, based on various morphological features such as: Area, Elongation, Euler number and other features. See table 1. The calculated features are, among others:

Candidate Area, Perimeter, aspect ratio, convex ratio, Number of holes & their respective area. For example “Minimal/Maximal blob area” is used to filter based on candidate area. Too narrow candidate will be removed with the Maximal Eccentricity & Maximal MajorAxisLengthT limiters.

TABLE 1
key parameters and heir operational range

| Name | Range | Explanation |
|---|---|---|
| Density resolution | 0-1 | Skin Complexion |
| | | detection |
| Density resolution | 0-1 | Boosting/suppressing |
| | | pixels |
| Combined Density resolution | 0-1 | Combined |
| | | Boosting/suppressing |
| | | pixels & skin saturation |
| Horizontal # of patches | 1-1024 | Rect roi size to calc hair |
| | | filter intensity |
| Vertical # of patches | 1-1024 | Rect roi size to calc hair |
| | | filter intensity |
| Hair Filter | | High pass Filter shape = |
| | | [a z1 b z2 c z3-c z4-b z5-a] |
| | | a, b, c - any number. Z1-Z5 |
| | | none/single zero |
| | | value or more for each Zi |
| | | value. |
| Color quantization level vertical | 8-1024 | Density estimation # |
| | | levels |
| Color quantization level horizontal | 8-1024 | Density estimation # |
| | | levels |
| # of clusters | 1-8 | To detect |
| Normalized Minimal clusters separation | 0-1 | If there are more than 1 |
| | | cluster |
| Normalized Minimal cluster significance | 0-1 | Otherwise noise - no |
| | | cluster |
| Savana feature Weight | 0.0-1.0 | Relative Weight of the |
| | | hair filter feature |
| Hair color Proximity feature Weight | 0.0-1.0 | Relative Weight of the |
| | | color feature |
| Edge Filters | | High pass Filter shape = |
| | | [a z1 b z2 c z3-c z4-b z5-a] |
| | | a, b, c - any number. Z1-Z5 |
| | | none/single zero |
| | | value or more for each Zi |
| | | value. |
| Horizontal # of Rect | 1-1024 | Rect roi size to calc edge |
| | | filter intensity |
| Vertical # of Rect | 1-1024 | Rect roi size to calc edge |
| | | filter intensity |
| Edge Std Threshold factor | −4 to +4 | Factor that helps to set |
| | | local segmentation |
| | | threshold on edge filter |
| Min Edge Threshold | 0-100 | Minimal allowed |
| | | Threshold value |
| Max Edge Threshold | 0-100 | Maximal allowed |
| | | Threshold value |
| Minimal/Maximal blob area | | Morphological |
| | | operations to clean, |
| | | smooth remove small |
| | | blobs & consolidate |
| | | blobs |
| Holes Area | 0-10000 | Conditional Fill of small |
| | | holes |
| Border Width & Height | 0-200 | Define border frame |
| | | width/Height |
| Minimal blob area | 0-10000 | To stay |
| Maximal blob area | 0-10000 | To stay |
| Small blobs filtering, the following actions: | All: | Morphological |
| 1. Expander | N = 0-10 | operations with filters in |
| 2. Eraser | A × B | order to clean, smooth |
| 3. Smoother | | remove small blobs |
| | | &amp; consolidate |
| | | blobs. |
| | | N = number of times to |
| | | operate. |
| | | Filters size: A × B, A, B any |
| | | number between 1-15. |
| | | Filters Values: every |
| | | coefficient can be any |
| | | number between 0 to 1, |
| | | Example A = 9, B = 1 filter |
| | | of the form [1, 1, 0.75, |
| | | 0.75, 0, 0.75, 0.75, 1, 1]. |
| Roi Margin Expansion width | −100 to 250 | Margin in order to calc 2 |
| | | objects density |
| Roi Margin Expansion height | −100 to 250 | Margin in order to calc 2 |
| | | objects density |
| Number of Thresholds/classes | 1-8 | To be segmented |
| Density resolution | 16-65000 | In order to estimate |
| | | density distribution |
| Minimal Threshold value | 0-10000 | To stay |
| Maximal blob area | 0-10000 | To stay |
| Minimal/Maximal blob area | | Morphological |
| | | operations to clean, |
| | | smooth remove small |
| | | blobs & consolidate |
| | | blobs |
| Holes Area | 0-10000 | Conditional Fill of small |
| | | holes |
| Maximal Eccentricity | 0-1 | Big Eccentricity - likely |
| | | to be artificial FP |
| Maximal MajorAxisLengthT | 1-100 | Big MajorAxisLength - |
| | | likely to be artificial FP |
| Maximal Euler number | 0-10 | # holes in segmented & |
| | | cleaned image |
| Maximal Convex ratio | 0-1 | Biological lesion is |
| | | convex shape |
| Minimal Convex Area | 0-1 | Biological lesion is |
| | | convex shape |
| Minimal normalized Extent | 0-1 | Biological lesion is |
| | | convex shape |
| Minimal normalized Solidity | 0-1 | Biological lesion is |
| | | convex shape |

FIGS. 13A-13B show two candidates: Candidate #106 (marked by a circle) is a mole while candidate #114 is a muscle wrinkle (marked by black square).

FIG. 14 shows the identification process of Candidate 106 of FIGS. 13A-13B.

FIG. 14A shows the input image. FIG. 14B show the segmentation result performed in the Approximate Localization step.

FIG. 14C shows the accurate segmentation result performed in this step.

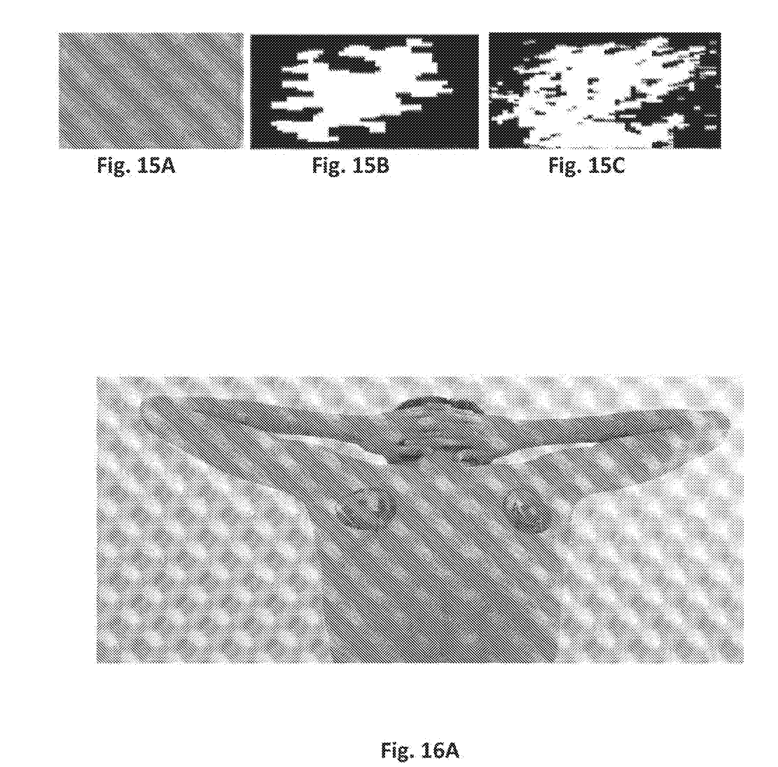

FIG. 15 shows the same identification process for Candidate 114 of FIGS. 13A-13B—wrinkle. The segmented blob, 15C, is morphology very different and hence will be filtered out.

Lastly FIG. 16A shows an outcome of all detected lesions. All detections are superimposed as green contours on the original image. See also the zoom in image in FIG. 16B. There are lots of TP as well as some FP and even a FN.

In some embodiments, the digital photograph is taken according to a total body photography (TBP) protocol. TBP or Whole Body Integumentary Photography is a well-established procedure for taking set of images that cover almost the entire body. These pictures are taken according to predefined set of body poses as can be seen in FIG. 18. The actual number of images taken can vary a little but it is usually around 25 pictures per person (range can be from 15 to 35 pictures per person). These sets include pictures taken from different angles, that is Front/Back/Left/Right side, covering the body from top to bottom. Additional images include feet, upper scalp and more as can be seen in the series of sectional photos in FIG. 18.

The system of the invention is capable to detect small lesions of size 0.5 millimeter (m) or higher.

Assuming:
H=the average height of a person, 170 cm (1,700 MM);
N=5, being the number of photographs needed to cover a body from feet to head in each of the 4 directions: front, back, left, right (total of 5*4=20 photographs per person);
W=3,000, length/width of a picture in pixels (2,000-5,000 range);
O=0.8, meaning 20% overlap between the photographs (range can be 0-30%)

The obtained resolution (R) can be calculated as:

R=W*N*O/H
R=3,000*5*0.8/1,700=7,058=about 7 pixels per MM.

The system of the invention can identify lesions (objects) of 3×3 pixels, since there are 7 pixels in a MM, the object of 3×3 pixels (length, width) is of length/width of 3/7=0.428 MM, or roughly 0.5 MM.

Although the invention has been described in detail, nevertheless changes and modifications, which do not depart from the teachings of the present invention, will be evident to those skilled in the art. Such changes and modifications are deemed to come within the purview of the present invention and the appended claims.

It will be readily apparent that the various methods and algorithms described herein may be implemented by, e.g., appropriately programmed general purpose computers and computing devices. Typically a processor (e.g., one or more microprocessors) will receive instruction from a memory or like device, and execute those instructions, thereby performing one or more processes defined by those instructions. Further, programs that implement such methods and algorithms may be stored and transmitted using a variety of media in a number of manners. In some embodiments, hard-wired circuitry or custom hardware may be used in place of, or in combination with, software instructions for implementation of the processes of various embodiments. Thus, embodiments are not limited to any specific combination of hardware and software.

A “processor” means any one or more microprocessors, central processing units (CPUs), computing devices, microcontrollers, digital signal processors, or like devices.

The term “computer-readable medium” refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprises a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviors of a database can be used to implement various processes, such as the described herein. In addition, the databases may, in a known manner, be stored locally or remotely from a device which accesses data in such a database.

The present invention can be configured to work in a network environment including a computer that is in communication, via a communications network, with one or more devices. The computer may communicate with the devices directly or indirectly, via a wired or wireless medium such as the Internet, LAN, WAN or Ethernet, Token Ring, or via any appropriate communications means or combination of communications means. Each of the devices may comprise computers, such as those based on the Intel®, Pentium®, or Centrino™, processor, that are adapted to communicate with the computer. Any number and type of machines may be in communication with the computer.

Appendix 1:

### List of Cutaneous Lesions and Other Clinically Interesting Objects (Non-Inclusive List)

| Lesions | Remarks |
|---|---|
| Moles | |
| Acne | |
| Nipple | |
| melanoma | |
| Gland Sabius | |
| Navel | |
| Hemangioma | |
| BGIHM/IHM | Benign guttate idiopathic hypomelanosis |
| Freckles | |
| Lentigos & solar lentigo | |
| Lentigo malinga | |
| Dermatofibroma | |
| sebaceos cyst | |
| BCC | Basal cell carcinoma |
| SCC | Squamous cell carcinoma |
| Seborrheic keratosis | |
| Merkel cell carcinoma | |
| Skin tags | |
| Benign nevus | compound, intra, dermal etc. |
| Angiokeratoma | |
| Fibrous papule | |
| Bite/wound | |
| wrinkle | |
| scars | |

1. A computing system comprising:
at least one processor; and
at least one memory communicatively coupled to the at least one processor comprising computer-readable instructions that when executed by the at least one processor cause the computing system to implement a method for analyzing a digital photograph comprising identified skin parts and analyzing cutaneous lesions, the method comprising the steps of:
(i) enhancing lesions in said identified skin parts, wherein said enhancing lesions comprises the steps of:
a. detecting skin complexion using common/averaged value of density estimation on a dominant channel extracted from skin pixels;
b. boosting lesions pixels by enhancement of lesion pixels and suppression of skin pixels; and
c. enhancing said Dominant Channel by combining said Dominant Channel with lesions boosting mechanism;
(ii) detecting hair patches;
(iii) approximating localization of all lesions; and
(iv) identifying lesions pixels.

2. The computing system according to claim 1, wherein said dominant channel is saturation, value, intensity, Red Green Blue (RGB) or any combination thereof.

3. The computing system according to claim 1, wherein detecting hair patches comprises the steps of:
(i) calculating one or more hair detection filters based on enhanced dominant channel (EDC);
(ii) calculating local normalized median or average on the filtered EDC;
(iii) calculating density estimation on the “value-EDC” planes or other planes;
(iv) detecting clusters;
(v) calculating how close is each cluster to hair color and skin color and assigning a hair color score to each cluster; and
(vi) assigning a patch hair probability score to each cluster based on each cluster's hair color score and savannah score.

4. The computing system according to claim 3, wherein detecting clusters is performed using semi-supervised k-means or spectral clustering or any other clustering method.

5. The computing system according to claim 1, wherein approximating localization of all lesions comprises the steps of:
(i) calculating one or more edge detection filters on EDC plane, said filters varying in length and coefficients values;
(ii) calculating of local median; average; median and standard deviation; or average and standard deviation on the filtered magnitude EDC image;
(iii) combining the results of step (i) and (ii) to create an automatic threshold setting for segmentation for each and every pixel on all regions and for every filter;
(iv) combining said various pixels outcomes and filters decisions to a objects candidates map;
(v) cleaning, smoothing and unifying objects based on filters and proximity;
(vi) filling small holes and gaps;
(vii) removing candidates that are not fully shown in a skin region or in entire image;
(viii) removing candidates that are too small, too narrow or too lacy; and
(ix) cleaning, smoothing and unifying objects again based on morphological filters.

6. The computing system according to claim 5, wherein said edge detection filters are of different shapes, sizes and structures based partly on patch hair probability scores.

7. The computing system according to claim 5, wherein said morphological filters are operations to clean, smooth and remove small blobs and consolidate blobs.

8. The computing system according to claim 5, wherein said morphological filters size is A*B, where A and B are a number between 1-15.

9. The computing system according to claim 1, wherein identifying of lesion pixels comprises performing the following steps for each lesion candidate:
(i) taking from image planes red/green/blue/value/EDC or any combination of one or more of said image planes the pixels that include the lesion candidate as well as its neighboring pixels;
(ii) performing density estimation and maximization of the inter class variation in order to get a suggested threshold for accurate segmentation;
(iii) verifying that the suggested threshold from (ii) is within a defined range;
(iv) perform thresholding, thus creating candidate objects;
(v) cleaning, smoothing and unifying objects based on morphological filters and proximity; and
(vi) fill small holes and gaps.

10. The computing system according to claim 9, further comprising the step of removing candidates based on one or more morphological features, wherein said one or more morphological features comprise: Area, Elongation, Euler number, Eccentricity, Major Axis Length, Convex ratio, Convex area, normalized Extent, Extent, normalized Solidity, Solidity.

11. (canceled)

12. The computing system according to claim 1, wherein said digital photograph was taken according to a total body photography protocol.

13. The computing system according to claim 1, wherein the lesions detected are of 0.5 millimeter (mm) or bigger.

14. A computer system comprising:
a processor; and
a memory communicatively coupled to the processor comprising computer-readable instructions that when executed by the processor cause the computer system to execute instructions for analyzing a digital photograph comprising identified skin parts and analyzing cutaneous lesions, the system comprising:
(i) an enhancement module adapted to enhancing via the processor lesions in said identified skin parts, wherein said enhancing lesions comprises the steps of:
a. detecting skin complexion using common/averaged value of density estimation on a dominant channel extracted from skin pixels, wherein said dominant channel is saturation, value, intensity, Red Green Blue (RGB) or any combination thereof;
b. boosting lesions pixels by enhancement of lesion pixels and suppression of skin pixels; and
c. enhancing said Dominant Channel by combining said Dominant Channel with lesions boosting mechanism;
(ii) a detection module adapted for detecting via the processor hair patches;
(iii) an approximation module adapted for approximating via the processor localization of all lesions; and
(iv) an identification module adapted for identifying via the processor lesions pixels.

15. (canceled)

16. The computer system according to claim 14, wherein said detection module is further adapted for:
(i) calculating one or more hair detection filters based on enhanced dominant channel (EDC);
(ii) calculating local normalized median or average on the filtered EDC;
(iii) calculating density estimation on the “value-EDC” planes or other planes;
(iv) detecting clusters;
(v) calculating how close is each cluster to hair color and skin color and assigning a hair color score to each cluster; and
(vi) assigning a patch hair probability score to each cluster based on each cluster's hair color score and savannah score.

17. The computer system according to claim 16, wherein detecting clusters is performed using semi-supervised k-means or spectral clustering or any other clustering method.

18. The computer system according to claim 14, wherein said approximation module is further adapted for:
(i) calculating one or more edge detection filters on EDC plane, said filters varying in length and coefficients values;
(ii) calculating of local median; average; median and standard deviation; or average and standard deviation on the filtered magnitude EDC image;
(iii) combining the results of step (i) and (ii) to create an automatic threshold setting for segmentation for each and every pixel on all regions and for every filter;
(iv) combining said various pixels outcomes and filters decisions to a objects candidates map;
(v) cleaning, smoothing and unifying objects based on filters and proximity;
(vi) filling small holes and gaps;
(vii) removing candidates that are not fully shown in a skin region or in entire image;
(viii) removing candidates that are too small, too narrow or too lacy; and
(ix) cleaning, smoothing and unifying objects again based on morphological filters.

19. The computer system according to claim 18, wherein said edge detection filters are of different shapes, sizes and structures based partly on patch hair probability scores.

20. The computer system according to claim 18, wherein said morphological filters are operations to clean, smooth and remove small blobs and consolidate blobs.

21. (canceled)

22. The computer system according to claim 14, wherein said identification module is further adapted to perform for each lesion candidate:
(i) taking from image planes red/green/blue/value/EDC or any combination of one or more of said image planes the pixels that include the lesion candidate as well as its neighboring pixels;
(ii) performing density estimation and maximization of the inter class variation in order to get a suggested threshold for accurate segmentation;
(iii) verifying that the suggested threshold from (ii) is within a defined range;
(iv) perform thresholding, thus creating candidate objects;
(v) cleaning, smoothing and unifying objects based on morphological filters and proximity; and
(vi) fill small holes and gaps.

23. The computer system according to claim 22, further adapted for removing candidates based on one or more morphological features, wherein said one or more morphological features comprise: Area, Elongation, Euler number, Eccentricity, Major Axis Length, Convex ratio, Convex area, normalized Extent, Extent, normalized Solidity, Solidity.

24. (canceled)

25. (canceled)

26. (canceled)
